(19) United States
(12) Patent Application Publication
(10) Pub. No.: US 2009/0093406 A1
(43) Pub. Date: Apr. 9, 2009

(54) Vegetable Protein Concentrate Having a Reduced Insoluble Dietary Fiber Content and Increase Amount of Soluble Dietary Fiber Content

(75) Inventors: Der-Chyan Hwang, Chesterfield, MO (US); Charles W. Monagle, St. Louis, MO (US); Traci R. Hamersen, Clayton, MO (US)

Correspondence Address: Solae, LLC, 4300 Duncan Avenue, Legal Department E4, St. Louis, MO, 63110, US

(73) Assignee: Solae, LLC, St. Louis, MO (US)

(21) Appl. No.: 12/207,092

(22) Filed: Sep. 9, 2008

Related U.S. Application Data
(60) Provisional application No. 60/971,141, filed on Sep. 10, 2007.

(51) Int. Cl. A61K 38/16; A23J 1/14; A23L 1/40; A21D 13/00; A23L 2/00; A23L 1/31

(52) U.S. Cl. 514/12; 426/656; 426/589; 426/580; 426/549; 426/590; 426/641

(57) ABSTRACT

This invention relates to a vegetable protein composition of matter derived from an alcohol washed vegetable protein material, comprising; having a decreased amount of insoluble dietary fiber content and an increased amount of soluble dietary fiber content relative to said original amount of insoluble dietary fiber and soluble dietary fiber in the alcohol washed vegetable protein material.

The invention further relates to processes for obtaining the vegetable protein composition having a decreased amount of insoluble dietary fiber content and an increased amount of soluble dietary fiber content.

### FIELD OF THE INVENTION

The present invention relates to a vegetable protein composition that has a reduced amount of insoluble dietary fiber and a corresponding increased amount of soluble dietary fiber, such that when incorporated into an emulsified meat, the protein-protein interaction of the soy protein and the meat protein improves the meat functionality. Further, the invention relates to dietary and therapeutic formulations that include the vegetable protein composition having a reduced amount of insoluble dietary fiber and a corresponding increased amount of soluble dietary fiber.

### BACKGROUND OF THE INVENTION

Fiber is known as a good water absorbent and has been widely used in food applications to help bind water. However, the greater hydration power of fiber interferes with meat texture since there is a softening of the hot dog texture due to the increase in water content. The goal is to change the form of fiber present in soy protein to be more compatible with the meat protein by decreasing the insoluble dietary fiber content and increasing soluble dietary fiber of the soy protein.

Plant protein materials are used as functional food ingredients, and have numerous applications in enhancing desirable characteristics in food products. Soy protein materials, in particular, have seen extensive use as functional food ingredients. Soy protein materials are used as meat extenders in meat products, such as frankfurters, sausages, bologna, ground and minced meats and meat patties, to bind the meat and give the meat a good texture and a firm bite. Another common application for soy protein materials as functional food ingredients is in creamed soups, gravies, and yogurts where the soy protein material acts as a thickening agent and provides a creamy viscosity to the food product. Soy protein materials are also used as functional food ingredients in numerous other food products such as dips, dairy products, tuna products, breads, cakes, macaroni, confections, whipped toppings, baked goods and many other applications.

Soy protein concentrates and soy protein isolates, which have relatively high concentrations of protein, are particularly effective functional food ingredients due to the versatility of soy protein. Soy protein provides gelling properties and has been used to modify the texture in ground and emulsified meat products. The texture-modifying gel structure provides dimensional stability to cooked meat emulsions which results in firm texture and desired chewiness. In addition, the gel structure provides a matrix for retaining moisture and fats.

Soy protein also acts as an emulsifier in various food applications since soy proteins are surface active and collect at oil-water interfaces, inhibiting the coalescence of fat and oil droplets. The emulsification properties of soy proteins allow soy protein containing materials to be used to thicken food products such as soups and gravies. The emulsification properties of soy protein materials also permit the soy protein materials to absorb fat and therefore promote fat binding in cooked foods so that “fatting out” of the fat during cooking processes can be limited. Soy protein materials also function to absorb water and retain it in finished food products due to the hydrophilic nature of the numerous polar side chains along the peptide backbone of soy proteins. The moisture retention of a soy protein material may be utilized to decrease cooking loss of moisture in a meat product, providing a yield gain in the cooked weight of the meat product. The retained water in the finished food products is also useful for providing a more tender mouthfeel to the product.

Soy protein based meat analog products or gelling food products, for example cheese and yogurt, offer many health benefits to consumers. Consumer acceptance of these products is directly related to organoleptic qualities such as texture, flavor, mouthfeel and appearance. Protein sources for gel-based food products such as meat analogs advantageously have good gel forming properties at relatively low cooking temperatures and good water and fat binding properties.

Both the strength of a gel and how it affects a final product into which it is to be incorporated are important considerations in determining the usefulness of a gel. The emulsification strength of a material is also an important characteristic to be considered in incorporating a material into a food product. As discussed above, the functionality of soy protein gels in food products and the emulsification properties of soy protein materials in food products have been well established.

Gel strengths of soy protein materials such as soy protein concentrates and soy protein isolates vary, and there is always a need for improvements in the gel strength of soy protein concentrates and isolates. Emulsification strengths of soy protein materials such as soy protein concentrates and soy protein isolates also vary, and there is always a need for improvements in the emulsification strength of soy protein materials such as soy protein concentrates and soy protein isolates. Especially desirable, particularly for use in emulsified meat products, are soy protein materials such as soy protein concentrates and soy protein isolates that have both strong gelling properties and strong emulsification properties.

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into blood plasma. High levels of total cholesterol (total-C), LDL-C, and apolipoprotein B (a membrane complex for LDL-C and VLDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C.

### SUMMARY OF THE INVENTION

The present invention provides a vegetable protein composition derived from an alcohol washed vegetable protein material, comprising; having a decreased amount of insoluble dietary fiber content of from about 28% up to about 75%, preferably up to about 65%, and most preferably up to about 55% by weight relative to said original amount of insoluble dietary fiber in the alcohol washed vegetable protein material.

Further, the vegetable protein composition of the present invention generally has an increased amount of soluble dietary fiber content of at least about 200% by weight relative to said original amount of soluble dietary fiber in the alcohol washed vegetable protein material.

The present invention also provides a food product comprising a blend of

a vegetable protein composition of matter derived from an alcohol washed vegetable protein material, having a decreased amount of insoluble dietary fiber content of from about 28% up to about 55% by weight relative to said original amount of insoluble dietary fiber in the alcohol washed vegetable protein material and

at least one food ingredient.

Further, the present invention also provides a food product comprising a blend of

a vegetable protein composition of matter derived from an alcohol washed vegetable protein material, wherein the alcohol washed vegetable protein material has an original amount of soluble dietary fiber, comprising; the vegetable protein composition of matter having a increased amount of soluble dietary fiber content of at about 200% by weight relative to said original amount of soluble dietary fiber in the alcohol washed vegetable protein material and

at least one food ingredient.

The present invention also provides a food product comprising a blend of

a vegetable protein composition of matter derived from an alcohol washed vegetable protein material, wherein the alcohol washed vegetable protein material has an original amount of insoluble dietary fiber, comprising; the vegetable protein composition of matter having a decreased amount of insoluble dietary fiber content of from about 34% up to about 75% by weight relative to said original amount of insoluble dietary fiber in the alcohol washed vegetable protein material and

at least one food ingredient.

Further, the present invention also provides a food product comprising a blend of

a vegetable protein composition of matter derived from an alcohol washed vegetable protein material, wherein the alcohol washed vegetable protein material has an original amount of soluble dietary fiber, comprising; the vegetable protein composition of matter having a increased amount of soluble dietary fiber content of at least about 200% by weight relative to said original amount of soluble dietary fiber in the alcohol washed vegetable protein material and

at least one food ingredient.

The present invention also provides a first process for obtaining a vegetable protein composition, comprising the steps of:

slurrying an alcohol washed vegetable protein material in water wherein the an alcohol washed vegetable protein material has an original amount of insoluble dietary fiber;

adjusting the pH of the slurry to between about 8.0 and about 11.0 to solubilize a portion of the insoluble dietary fiber to provide a pH adjusted slurry;

adjusting the pH of the slurry to less than about 6.0 to precipitate protein;

removing soluble components from the acid pH slurry to provide a non-soluble curd;

adding water to the curd to provide a washed curd slurry; adjusting the pH of the washed curd slurry to about 7.0 to provide a neutralized slurry; and

subjecting the neutralized slurry to heat treatment at a sufficient temperature and for a sufficient period of time to change the structure of the protein to provide a heat treated slurry;

wherein the vegetable protein composition of matter has a decreased amount of insoluble dietary fiber content of from about 28% up to about 55% by weight relative to said original amount of insoluble dietary fiber in the alcohol washed vegetable protein material.

In this first process, the vegetable protein composition has an increased amount of soluble dietary fiber content of at least about 200% by weight relative to the original amount of soluble dietary fiber in the alcohol washed vegetable protein material.

The present invention also provides a second process for obtaining a vegetable protein composition, comprising the steps of:

slurrying an alcohol washed vegetable protein material in water wherein the alcohol washed vegetable protein material has an original amount of insoluble dietary fiber;

adjusting the pH of the slurry to between about 7.4 and about 9.0 to solubilize a portion of the insoluble dietary fiber to provide a pH adjusted slurry;

subjecting the pH adjusted slurry to heat treatment at a sufficient temperature and for a sufficient period of time to change the structure of the protein to provide a heat treated pH adjusted slurry; and

adjusting the pH of the heat treated pH adjusted slurry to about 7.0 to provide a neutralized slurry;

wherein the vegetable protein composition of matter has a decreased amount of insoluble dietary fiber content of from about 34% up to about 75% by weight relative to said original amount of insoluble dietary fiber in the alcohol washed vegetable protein material.

In this second process, the vegetable protein composition has an increased amount of soluble dietary fiber content of at least about 200% by weight relative to the original amount of soluble dietary fiber in the alcohol washed vegetable protein material.

The present invention also provides a method for treating at least one of the following diseases, disorders, or conditions selected from the group consisting of cardiovascular disease, hypercholesterolemia disorder, low serum high density lipid (HDL)/low density lipid (LDL) ratio, hypertriglyceridemia disorder, diabetes, and weight loss in a human comprising administering to a human in need thereof an effective amount of the vegetable protein composition, wherein the vegetable protein composition is derived from an alcohol washed vegetable protein material, wherein the alcohol washed vegetable protein material has an original amount of insoluble dietary fiber, comprising; the vegetable protein composition of matter having a decreased amount of insoluble dietary fiber content of from about 28% up to about 75%, preferably up to about 65%, and most preferably up to about 55% by weight relative to said original amount of insoluble dietary fiber in the alcohol washed vegetable protein material.

Further, the vegetable protein composition of matter is derived from an alcohol washed vegetable protein material, wherein the alcohol washed vegetable protein material has an original amount of soluble dietary fiber, comprising; the vegetable protein composition of matter having a increased amount of soluble dietary fiber content of at least about 200% by weight relative to said original amount of soluble dietary fiber in the alcohol washed vegetable protein material.

Further, the present invention provides a method for treating or offsetting the risks of a disease or disorder such as cardiovascular disease, hypercholesterolemia disorder, low serum high density lipid (HDL)/low density lipid (LDL) ratio, hypertriglyceridemia disorder, diabetes, and weight loss in a human comprising administering to a human in need thereof an effective amount of the above vegetable protein composition. The method includes the step of administering to a human in need thereof a food that includes an effective amount of a vegetable protein composition of matter derived from an alcohol washed vegetable protein material, wherein the alcohol washed vegetable protein material has an original amount of insoluble dietary fiber, comprising; the vegetable protein composition of matter having a decreased amount of insoluble dietary fiber content of from about 28% up to about 75%, preferably up to about 65%, and most preferably up to about 55% by weight relative to said original amount of insoluble dietary fiber in the alcohol washed vegetable protein material.

Further, the vegetable protein composition of matter is derived from an alcohol washed vegetable protein material, wherein the alcohol washed vegetable protein material has an original amount of soluble dietary fiber, comprising; the vegetable protein composition of matter having a increased amount of soluble dietary fiber content of at least about 200% by weight relative to said original amount of soluble dietary fiber in the alcohol washed vegetable protein material.

Further, the present invention provides a method for inducing satiety in a human. The method includes the step of administering to a human in need thereof a food that includes an effective amount of a vegetable protein composition of matter derived from an alcohol washed vegetable protein material, wherein the alcohol washed vegetable protein material has an original amount of insoluble dietary fiber, comprising; the vegetable protein composition of matter having a decreased amount of insoluble dietary fiber content of from about 28% up to about 75%, preferably up to about 65%, and most preferably up to about 55% by weight relative to said original amount of insoluble dietary fiber in the alcohol washed vegetable protein material.

Further, the vegetable protein composition of matter is derived from an alcohol washed vegetable protein material, wherein the alcohol washed vegetable protein material has an original amount of soluble dietary fiber, comprising; the vegetable protein composition of matter having a increased amount of soluble dietary fiber content of at least about 200% by weight relative to said original amount of soluble dietary fiber in the alcohol washed vegetable protein material.

The vegetable protein material for the vegetable protein composition of the present invention is selected from the group consisting of a soy protein material, a canola protein material, and a corn protein material. The soy protein material may be derived from commodity soybeans, genetically modified soybeans, high beta-conglycinin soybeans, and high oleic soybeans. Preferred are protein materials derived from soybeans.

### BRIEF DESCRIPTION OF THE DRAWINGS

FIG. 1 is a graph of the water holding capacity (WHC) of an emulsified meat composition versus the percent reduction in insoluble dietary fiber (IDF) of a soy protein composition prepared by the first process.

FIG. 2 is a graph of the water holding capacity (WHC) of an emulsified meat composition versus the percent reduction in insoluble dietary fiber (IDF) of a soy protein composition prepared by the second process.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention also relates generally to soy protein compositions that includes a decreased amount of insoluble fiber content, with a corresponding increased amount of soluble dietary fiber content that are capable of facilitating weight loss and/or improving cardiovascular health by lowering serum cholesterol concentrations, lowering serum triglyceride concentrations, raising HDL/LDL cholesterol ratios, reducing blood glucose levels after meals, stabilizing blood glucose levels between meals, lowering inflammatory mediator concentrations, and lowering blood pressure in a human. Further, the present invention also relates generally to soy protein compositions that includes a increased amount of soluble fiber content, with a corresponding decreased amount of insoluble dietary fiber content that are capable of facilitating weight loss and/or improving cardiovascular health by lowering serum cholesterol concentrations, lowering serum triglyceride concentrations, raising HDL/LDL cholesterol ratios, reducing blood glucose levels after meals, stabilizing blood glucose levels between meals, lowering inflammatory mediator concentrations, and lowering blood pressure in a human. Such formulations also have positive effects on measurable parameters of oxidative stress, inflammation and aging.

Cardiovascular disorders and other health conditions such as those listed above may be signs of disease processes and are conventionally treated by administering suitable drugs. The soy protein composition of the present invention is used to supplant the need for such drugs. According to specific embodiments of the invention, the soy protein composition is used to reduce the daily requirement of such drugs to adequately control disease conditions such as weight loss, hypercholesterolemia, hyperlipidemia, hypertension, diabetes, prediabetic syndrome in adults and children, and diabetic predisposition in adults and children.

Hyperlipidemia is a known risk factor for the development of atherosclerosis, coronary artery disease, and myocardial infarction. Hypercholesterolemia is a pathogenic factor for arteriosclerosis, stroke, heart disease, and angina pectoris. Consequently, reducing plasma lipid concentrations, serum cholesterol concentrations, and serum triglyceride concentrations is a priority for combating the onset and progression of cardiovascular disease. Plasma lipid reduction is routinely accomplished by employing dietary modifications and/or by intake of pharmaceutical drugs such as the statin family of drugs, which inhibit the synthesis of cholesterol in the liver.

Diabetes and obesity are common ailments in the United States and other Western cultures. A study by researchers at RTI International and the Centers for Disease Control estimated that U.S. obesity-attributable medical expenditures reached $75 billion in 2003. Obesity has been shown to promote many chronic diseases, including type 2 diabetes, cardiovascular disease, several types of cancer, and gallbladder disease.

Adequate dietary intake of soluble fiber has been associated with a number of health benefits, including decreased blood cholesterol levels, improved glycemic control, and the induction of satiety and satiation in individuals. Consumers have been resistant to increasing soluble fiber amounts in their diet, however, often due to the negative organoleptic characteristics, such as, sliminess, excessive viscosity, excessive dryness and poor flavor, that are associated with food products that include soluble fiber.

### DEFINITIONS

The use of the term “fiber” is often used as a catchall name for various fibrous fractions such as “crude fiber” and “dietary fiber”. Crude fiber is generally understood to mean the residue left after boiling the food in dilute caustic and then in dilute acid. This method recovers about 50-80% of cellulose, about 10-50% of lignin and about 20% of hemicellulose. Generally, for purposes of the present invention the term “fiber” or “dietary fiber” is intended to mean any food which when ingested in a monogastric animal, especially a human, reaches the large intestine essentially unchanged. In essence, fiber is understood to mean those constituents derived from botanical materials which are resistant to human digestive enzymes.

Dietary fiber is more particularly defined as the sum of all polysaccharides and lignin that are not digested by the endogenous secretions of the human digestive tract. The polysaccharides are derived from either the plant cell-wall or cell-content. Those carbohydrates which are contained in the plant cell-wall include gums, mucilages, pectins, pectin substances, algal polysaccharides and hemicelluloses. All of these carbohydrate materials are classified as polysaccharides. Thus, for purposes of this invention, fiber and dietary fiber includes the above polysaccharides in addition to cellulose and lignin, individually or in combination, derived from one or more plant varieties or species. Although the term “fiber” commonly is used to refer to filamentous string-like materials, dietary fiber is generally gelatinous or mucilaginous in character.

As used herein, the term “total dietary fiber” or “dietary fiber” refers to the sum of the soluble dietary fiber (SDF) and insoluble dietary fiber (IDF). These food components are not broken down by the alimentary enzymes of humans to small molecules which are absorbed into the bloodstream. Increasing soluble dietary fiber intake improves intestinal and overall health by providing nutrients to intestinal flora. Insoluble dietary fiber promotes overall health by providing indigestible bulk to food products. However, the addition of high levels of fiber, particularly insoluble fiber, to food products is known to adversely affect the organoleptic properties of these food products. High fiber food products can have a dry, tough, chewy, or dense texture, making them less appealing to consumers.

“Soluble” and “insoluble” dietary fiber are determined using American Association of Cereal Chemists (AACC) Method 32-07. A “soluble” dietary fiber source refers to a fiber source in which at least 60% of the dietary fiber is soluble dietary fiber as determined by AACC Method 32-07, and an “insoluble” dietary fiber source refers to a fiber source in which at least 60% of the total dietary fiber is insoluble dietary fiber as determined by AACC Method 32-07.

As used herein, the term “soy material” is defined as a material derived from whole soybeans which contain no non-soy derived additives. Such additives may, of course, be added to a soy material to provide further functionality either to the soy material or to a food in which the soy material is utilized as a food ingredient. The term “soybean” refers to the species Glycine max, Glycine soja, or any species that is sexually cross compatible with Glycine max.

As used herein, the term “soy protein material” refers to a soy protein containing material that contains at least 40% soy protein by weight on a moisture-free basis.

As used herein, the term “soy protein concentrate” refers to soy protein containing material that contains from 65% up to 90% soy protein by weight on a moisture free basis.

The term “protein content” as used herein, refers to the relative protein content of a soy material as ascertained by A.O.C.S. (American Oil Chemists Society) Official Methods Bc 4-91 (1997), Aa 5-91 (1997), or Ba 4d-90 (1997), each incorporated herein in its entirety by reference, which determine the total nitrogen content of a soy material sample as ammonia, and the protein content as 6.25 times the total nitrogen content of the sample. The Nitrogen-Ammonia-Protein Modified Kjeldahl Method of A.O.C.S. Methods Bc4-91 (1997), Aa 5-91 (1997), and Ba 4d-90 (1997) used in the determination of the protein content may be performed as follows with a soy material sample. A sample of 0.0250-1.750 grams of the soy material are weighed into a standard Kjeldahl flask. A commercially available catalyst mixture of 16.7 grams potassium sulfate, 0.6 grams titanium dioxide, 0.01 grams of copper sulfate, and 0.3 grams of pumice is added to the flask, then 30 milliliters of concentrated sulfuric acid is added to the flask. Boiling stones are added to the mixture, and the sample is digested by heating the sample in a boiling water bath for approximately 45 minutes. The flask should be rotated at least 3 times during the digestion. Three hundred milliliters of water is added to the sample, and the sample is cooled to room temperature. Standardized 0.5N hydrochloric acid and distilled water are added to a distillate receiving flask sufficient to cover the end of a distillation outlet tube at the bottom of the receiving flask. Sodium hydroxide solution is added to the digestion flask in an amount sufficient to make the digestion solution strongly alkaline. The digestion flask is then immediately connected to the distillation outlet tube, the contents of the digestion flask are thoroughly mixed by shaking, and heat is applied to the digestion flask at about a 7.5-min boil rate until at least 150 milliliters of distillate is collected. The contents of the receiving flask are then titrated with 0.25N sodium hydroxide solution using 3 or 4 drops of methyl red indicator solution—0.1% in ethyl alcohol. A blank determination of all the reagents is conducted simultaneously with the sample and similar in all respects, and correction is made for blank determined on the reagents. The moisture content of the ground sample is determined according to the procedure described below (A.O.C.S Official Method Ba 2a-38). The nitrogen content of the sample is determined according to the formula: Nitrogen (%) 1400.67×[[(Normality of standard acid)×(Volume of standard acid used for sample (ml))]−[(Volume of standard base needed to titrate 1 ml of standard acid minus volume of standard base needed to titrate reagent blank carried through method and distilled into 1 ml standard acid (ml))×(Normality of standard base)]−[(Volume of standard base used for the sample (ml))×(Normality of standard base)]]/(Milligrams of sample). The protein content is 6.25 times the nitrogen content of the sample.

The term “moisture content” as used herein refers to the amount of moisture in a material. The moisture content of a soy material can be determined by A.O.C.S. (American Oil Chemists Society) Method Ba 2a-38 (1997), which is incorporated herein by reference in its entirety. According to the method, the moisture content of a soy material may be measured by passing a 1000 gram sample of the soy material through a 6×6 riffle divider, available from Seedboro Equipment Co., Chicago, Ill., and reducing the sample size to 100 grams. The 100 gram sample is then immediately placed in an airtight container and weighed. Five grams of the sample are weighed onto a tared moisture dish (minimum 30 gauge, approximately 50×20 millimeters, with a tight-fitting slip cover—available from Sargent-Welch Co.). The dish containing the sample is placed in a forced draft oven and dried at 130±3° C. (261°-271° F.) for 2 hours. The dish is then removed from the oven, covered immediately, and cooled in a desiccator to room temperature. The dish is then weighed. Moisture content is calculated according to the formula: Moisture content (%)=100×[(loss in mass (grams)/mass of sample (grams)].

The term “soy flour” as used herein means a soy protein material that is particulate and contains less than 65% soy protein content by weight on a moisture free basis which is formed from dehulled soybeans and which has an average particle size of 150 microns or less. A soy flour may contain fat inherent in soy or may be defatted.

The term “soy grit” as used herein means a soy protein material that is particulate and contains less than 65% soy protein content by weight on a moisture free basis which is formed from dehulled soybeans and which has an average particle size of from 150 microns to 1000 microns. A soy grit may contain fat inherent in soy or may be defatted.

The term “soy meal” as used herein means a soy protein material that is particulate and contains less than 65% soy protein content by weight on a moisture free basis which is formed from dehulled soybeans which does not fall within the definition of a soy flour or a soy grit. The term soy meal is intended to be utilized herein as a catchall for particulate soy protein containing materials having less than 65% protein on a moisture free basis which do not fit the definition of a soy flour or a soy grit. A soy meal may contain fat inherent in soy or may be defatted.

The term “soy flakes” as used herein means a soy protein material that is a flaked soy material containing less than 65% soy protein content by weight on a moisture free basis formed by flaking dehulled soybeans. Soy flakes may contain fat inherent in soy or may be defatted.

The “term “nitrogen solubility index” as used herein is defined as: (% water soluble nitrogen of a protein containing sample divided by the % total nitrogen in protein containing sample)×100. The nitrogen solubility index provides a measure of the percent of water soluble protein relative to total protein in a protein containing material. The nitrogen solubility index of a soy material is measured in accordance with standard analytical methods, specifically A.O.C.S. Method Ba 11-65, which is incorporated herein by reference in its entirety. According to the Method Ba 11-65, 5 grams of a soy material sample ground fine enough so that at least 95% of the sample will pass through a U.S. grade 100 mesh screen (average particle size of less than about 150 microns) is suspended in 200 milliliters of distilled water, with stirring at 120 revolutions per minute, at 30° C. (86° F.) for two hours, and then is diluted to 250 milliliters with additional distilled water. If the soy material is a full-fat material the sample need only be ground fine enough so that at least 80% of the material will pass through a U.S. grade 80 mesh screen (approximately 175 microns), and 90% will pass through a U.S. grade 60 mesh screen (approximately 205 microns). Dry ice should be added to the soy material sample during grinding to prevent denaturation of sample. 40 milliliters of the sample extract is decanted and centrifuged for 10 minutes at 1500 revolutions per minute, and an aliquot of the supernatant is analyzed for Kjeldahl protein (PRKR) to determine the percent of water soluble nitrogen in the soy material sample according to A.O.C.S Official Methods Bc 4-91 (1997), Ba 4d-90, or Aa 5-91, as described above. A separate portion of the soy material sample is analyzed for total protein by the PRKR method to determine the total nitrogen in the sample. The resulting values of Percent Water Soluble Nitrogen and Percent Total Nitrogen are utilized in the formula above to calculate the nitrogen solubility index.

The term “weight on a moisture free basis” as used herein refers to the weight of a material after it has been dried to completely remove all moisture, e.g. the moisture content of the material is 0%. Specifically, the weight on a moisture free basis of a soy material can be obtained by weighing the soy material after the soy material has been placed in a 45° C. (113° F.) oven until the soy material reaches a constant weight.

As used herein, the term “water-holding capacity” is defined as the maximum amount of water a material can absorb and retain under application of external forces, such as heating, cutting, mincing, pressing, and low speed centrifugation.

In spite of the difficulty to differentiate exactly the different forms of water bound or retained in a protein-rich food system, the following definition will be made. Generally, the water held in a protein structure can be divided into two main types: 1) that part bound to the molecule and is no longer available as a solvent and 2) the other part trapped in the protein matrix or a corresponding co-matrix (polysaccharide, fat). The first type can be regarded as absorbed water and the second as retained water. In most cases, the water-holding capacity of a protein matrix is determined by both the amount of absorbed and retained water.

The absorbed water, which is more tightly bound to the protein molecules, will be considered first. This type of water is largely influenced by the physicochemical parameters that directly affect the proteins and the surface properties of the protein molecules that interact with the dissolving solution. This means that the water-holding capacity depends not only on pore and capillary size but also oil the charges of the protein molecules (hydrophobic interactions, hydrogen bonds, S—S bonds, acids, bases, and zwitterions) as well as on Van der Waals' forces. In addition to these parameters, the surrounding medium may also affect the protein due to ionic strength, ion species, pH condition, temperature, and the time taken for equilibrating the protein with the water. In particular, low molecular weight substances (lactose and mineral salts such as sodium chloride) are reported to have a significant effect on the water-holding capacity of some proteins.

Retained water, on the other hand, is influenced by different structures that establish networks that immobilize water. This water should not be considered as free water. Free water is more commonly associated with the final product and means that it is retained by a co-matrix that enables or contributes to gel formation. Several substances (mainly proteins, are known to be capable of forming such gels, which can absorb and retain a substantial amount of water. This special feature may be added to certain foods such as processed cheese, cheese analogues, meat and fish products, pasties, baked goods, and also to various nonfood products (e.g., pharmaceuticals, paints, concrete, etc.) by incorporating the substances into the matrices of varying degrees of complexity.

The vegetable protein material for the vegetable protein composition of the present invention is selected from the group consisting of a soy protein material, a canola protein material, and a corn protein material. The soy protein material may be derived from commodity soybeans, genetically modified soybeans, high beta-conglycinin soybeans, and high oleic soybeans. Preferred are protein materials derived from soybeans.

Protein concentrates produced from commodity soybeans generally have a beta-conglycinin content of 26-29% of the soy protein content and a glycinin content of about 40-45%. Commodity soybeans generally have an oleic acid content of about 23%.

The term high beta-conglycinin concentrate refers to a soy protein concentrate in which beta-conglycinin content is greater than 40% of the soy protein content and the glycinin content is less than 10% of the soy protein content.

Soybean proteins are composed of four globulin fractions: 2S having a molecular weight from about 8,000 to about 21,500; 7S (beta-conglycinin) having a molecular weight from about 150,000 to about 200,000; 11S (glycinin) having a molecular weight of about 320,000 to about 380,000 and 15S having a molecular weight of about 600,000 to about 700,000.

The soy protein concentrate utilized within the present invention may have a beta conglycinin content of from about 40% to about 85% of the total weight of the soy protein and a glycinin content of from about 5% to about 40% of the total weight of the soy protein. The use of high beta conglycinin soybeans which contain more than about 40% beta conglycinin, enable the preparation of a soy protein concentrate having a beta conglycinin content of from about 40% to about 85% without the inefficiencies of removing glycinins during processing. The high beta conglycinin soy protein concentrate of the present invention contains from about 40% to about 85% beta conglycinin, compared to 26-29% in commercial soy protein concentrates. Typically soy protein concentrates contain about 40-45% glycinin. The high beta conglycinin soy protein concentrate of the present invention contains less than about 40% glycinin.

The term “GMO-free” refers to a composition that has been derived from a process in which genetically modified organisms are not utilized.

A genetically modified organism (GMO) is an organism whose genetic material has been altered using genetic engineering techniques generally known as recombinant DNA technology. Recombinant DNA technology is the ability to combine DNA molecules from different sources into one molecule in vitro. Thus, the expression of certain traits, the phenotype of the organism, or the proteins it produces, can be altered through the modification of its genes.

The term generally does not cover organisms whose genetic makeup has been altered by conventional cross breeding or by “mutagenesis” breeding, as these methods predate the discovery of the recombinant DNA techniques.

The soybean source may be a high oleic soybean. The term “high oleic” refers to an elevated oleic acid content of the soybean oil derived from the soybeans. A typical fatty acids profile of soybean oil derived from commodity soybeans is comprised primarily of saturated (13%), mono unsaturated as oleic (23%), and polyunsaturated (62%) acids. A fatty acids profile of soybean oil derived from high oleic soybeans is comprised primarily of saturated (10%), mono unsaturated as oleic (80%), and polyunsaturated (8%) acids. Soy protein concentrates derived from commodity soybeans have a residual triglyceride fatty acid content of a specific profile as defined above. A soy protein concentrate derived from a high oleic soybean has a residual triglyceride fatty acid content of primarily mono unsaturation in the protein. This latter protein is much more oxidatively stable due to the increase in the residual mono unsaturation level of the protein.

### Process

There are several processes for obtaining the vegetable protein composition of the present invention. Preferably, the vegetable protein composition is a soy protein composition. The starting material is the same for all processes. The starting material is an alcohol washed soy protein concentrate. Alcohol washed soy protein concentrates, sometimes known in the art as “traditional” soy protein concentrates, are commercially available from many sources. One alcohol washed soy protein concentrate which is suitable as a starting material for the present invention is Procon® 2000 soy protein concentrate, which is available from The Solae Company of St. Louis, Mo. Another suitable commercially available alcohol washed soy protein concentrate is Danpro H® soy protein concentrate, also available from The Solae Company.

It is to be understood that rather than use a commercially available alcohol washed soy protein concentrate as the starting material in the present invention, the starting material can be soy flour, soy grits, soy meal, or soy flakes from which an alcohol washed soy protein concentrate can be produced using by washing the soy flour, soy grits, soy meal, or soy flakes with a low molecular weight aqueous alcohol, preferably aqueous ethanol, followed by desolventizing the alcohol washed soy protein material. Soy flour, soy grits, soy meal, or soy flakes are commercially available, or, alternatively, may be produced from soybeans according to processes well known in the art. The thus produced alcohol washed soy concentrate can then be used in the processes as described herein.

In a first process for obtaining the soy protein composition of the present invention, the steps of comprise:

slurrying an alcohol washed soy protein material in water wherein the an alcohol washed soy protein material has an original amount of insoluble dietary fiber;

adjusting the pH of the slurry to between about 8.0 and about 11.0 to solubilize a portion of the insoluble dietary fiber to provide a pH adjusted slurry;

adjusting the pH of the slurry to less than about 6.0 to precipitate protein;

removing soluble components from the acid pH slurry to provide a non-soluble curd;

adding water to the curd to provide a washed curd slurry;

adjusting the pH of the washed curd slurry to about 7.0 to provide a neutralized slurry; and

subjecting the neutralized slurry to heat treatment at a sufficient temperature and for a sufficient period of time to change the structure of the protein to provide a heat treated slurry;

wherein the soy protein composition of matter has a decreased amount of insoluble dietary fiber content of from about 28% up to about 55% by weight relative to said original amount of insoluble dietary fiber in the alcohol washed soy protein material.

Further, the soy protein composition has an increased amount of soluble dietary fiber content of at least about 200% by weight relative to the original amount of soluble dietary fiber in the alcohol washed soy protein material.

In the first process, the alcohol washed soy protein concentrate is first slurried with water at a solids content of from about 1.0 wt. % to about 15.0 wt. %. Preferably, the alcohol washed soy protein concentrate is slurried with water at a solids content of from about 5.0 wt. % to about 12.0 wt. %. The water used to slurry the soy protein concentrate is preferably heated to a temperature of about 27° C. about to 82° C. (81° F.-180° F.).

The pH of the slurry is adjusted to between about 8.0 up to about 11.0 in order to solubilize the insoluble dietary fiber in the slurry. In a preferred embodiment, the pH is adjusted to between about 8.0 up to about 10.5, and most preferably between about 9.0 up to about 10.0. The pH of the slurry can be adjusted by addition of a suitable basic material. Most preferred are potassium hydroxide and sodium hydroxide.

After solubilization of the fiber by pH adjustment at the pH of from about 8.0 up to about 11, the pH of the slurry is then adjusted to less than about 6.0. The acid addition causes the formation of curds and whey. This pH adjustment causes components such as minerals to become soluble (whey) while minimizing protein solubility (curds) to facilitate removal of the soluble components. Further some, but not all of the insoluble dietary fiber that was made soluble with the basic material reverts and again becomes insoluble dietary fiber upon the pH adjustment to less than about 6.0. In a preferred embodiment, the pH is adjusted to between about 4.3 and about 5.3, preferably between about 5.0 and about 5.2, or, to about the isoelectric point of soy protein which is between pH about 4.4 and about 4.6. The pH of the slurry can be adjusted by addition of hydrochloric acid or other suitable edible organic or inorganic acid.

After pH adjustment, the slurry is subjected to a separation process. The separation process assists with the removal of soluble components. Suitable processes for removing soluble components include centrifugation, and other conventional separation processes. Alcohol washing to produce the alcohol washed soy protein removes large amounts of “soy solubles”. As such, it is unexpected that further removal of solubles would affect the characteristics of a soy protein material already washed with alcohol since it would be expected that the alcohol wash would have removed a large majority of such solubles.

According to one embodiment of the present invention, the slurry is subjected to a centrifugation separation process to remove the whey and leave behind the curd. A preferred centrifuge is a decanting centrifuge. The soluble components are removed in the liquor fraction, while insoluble materials such as the soy protein are retained in the insoluble cake of the centrifuge. Optionally, the centrifuge process may be repeated one or more times, in which the centrifuge cake of a first centrifugation is diluted with water and then is centrifuged again.

The slurry retained after removing the soluble components by the above separation processes has an increase in protein content and has a reduced ash content due to removal of the minerals. The obtained curd centrifuge cake is water washed one or more times to remove trace solubles from the centrifuge cake. In this instance, the centrifuge cake is diluted to make a slurry of from 7.0 wt. % to 20 wt. % solids, preferably from 10.0 wt. % to 15.0 wt. % solids, and most preferably from 12 wt. % to 13 wt. % solids.

After removing the solubles, the pH of the slurry is adjusted to 7.0 or more in order to neutralize the slurry, thereby increasing the solubility of the protein in the slurry. In one embodiment, the pH is adjusted to a pH of from 7.0 to 7.5, where pH 7.2 has been found to be particularly suitable. The pH of the slurry can be adjusted by addition of any suitable organic or inorganic base, preferably sodium hydroxide.

To produce a soy protein concentrate composition in accordance with the present invention, the resulting pH adjusted slurry is subjected to a heat treatment or cooking process, and optionally to a shearing process, to change the protein structure and to yield a final product that can optionally be dried.

The heat treatment or cooking process and the optional shearing process changes the structure of the protein to improve the functionality of the protein, producing a product that has high gel strength. Although any cooking process or apparatus can be used provided the soy protein material is subjected to sufficient heat for a sufficient period of time to change the structure of the soy protein material, jet cooking is deemed to be particularly suitable for commercial production of the soy protein concentrate of the present invention. Preferably the neutralized slurry of soy protein material is treated at a temperature of from about 75° C. to about 180° C. (167′-356° F.) for a period of from about 2 seconds to about 2 hours to change the structure of the soy protein in the soy protein material, where the soy protein material slurry is heated for a longer time period at lower temperatures to change the structure of the soy protein in the soy protein material. Preferably, the neutralized slurry is heat treated at a temperature of from 135° C. to 180° C. (275′-356° F.) for a period of 5 to 30 seconds, and most preferably the slurry is heat treated at a temperature of from 145° C. to 155° C. (293′-311° F.) for a period of from 5 to 15 seconds. Most preferably the soy protein material slurry is treated at an elevated temperature and under a positive pressure greater than atmospheric pressure.

As noted above, the preferred method of heat treating the soy protein material slurry is jet-cooking, which consists of injecting pressurized steam into the slurry to heat the slurry to the desired temperature. The following description is a preferred method of jet-cooking the soy protein material slurry, however, the invention is not limited to the described method and includes any obvious modifications which may be made by one skilled in the art.

The soy protein material slurry is introduced into a jet-cooker feed tank where the soy protein material is kept in suspension with a mixer which agitates the soy protein material slurry. The slurry is directed from the feed tank to a pump that forces the slurry through a reactor tube. Steam is injected into the soy protein material slurry under pressure as the slurry enters the reactor tube, instantly heating the slurry to the desired temperature. The temperature is controlled by adjusting the pressure of the injected steam, and preferably is from about 75° C. to about 180° C. (167′-356° F.), more preferably from about 135° C. to 180° C. (275′-356° F.).

After jet cooking, the slurry is held at a high temperature for a period of from 5 seconds to 240 seconds. A total holding time of from 30 seconds to 180 seconds is particularly suitable for the purposes of the present invention.

After cooking, preferably prior to holding the slurry at high temperature, the slurry is optionally subjected to a shearing process to maintain homogeneity. Any suitable shearing equipment can be used such as shearing pumps, shearing mixers, or cutting mixers. One suitable shear pump is a Dispax Reactor dispersing pump with three stages (IKA Works, Wilmington, N.C.). These pumps may be equipped with coarse, medium, fine and superfine generators. Each generator consists of a stator and a rotor. A preferred embodiment is to use two fine generators and a superfine generator in the three stages of the pump. Another suitable pump is a high pressure homogenizer. Other shear pumps are commercially available from Fristam Pumps Inc., Middleton, Wis. and Waukesha Cherry-Burrell, Delavan, Wis.

After cooking, optionally shearing the soy protein material, and holding the heated slurry at a high temperature, the slurry is then cooled. Preferably the slurry is flash cooled to a temperature of from 60° C. to 93° C. (140° F.-200° F.), and most preferably flash cooled to a temperature of from 80° C. to 90° C. (176°-194° F.). The slurry is flash cooled by introducing the heated slurry into a vacuumized chamber having a cooler internal temperature than the temperature used to heat treat the soy protein material slurry and having a pressure significantly less than atmospheric pressure. Preferably the vacuum chamber has an internal temperature of from 15° C. to 85° C. (59° F.-185° F.) and a pressure of from about 25 mm to about 100 mm Hg, and more preferably a pressure of from about 25 mm Hg to about 30 mm Hg. Introduction of the heated soy protein material slurry into the vacuum chamber instantly drops the pressure around the soy protein material slurry causing vaporization of a portion of the water from the slurry thereby cooling the slurry.

Flash cooling is the preferred cooling process, although it may be replaced by any other suitable cooling process which is capable of reducing the temperature to between about 60° C. and about 93° C. (140-200° F.) in a short period of time.

The cooled slurry of the soy protein composition may then be dried to produce the powdered soy protein concentrate composition of the present invention. The cooled slurry is preferably spray-dried to produce the soy protein material composition of the present invention. The spray-dry conditions should be moderate to avoid further denaturing the soy protein in the soy protein composition. Preferably the spray-dryer is a co-current flow dryer where hot inlet air and the soy protein material slurry, atomized by being injected into the dryer under pressure through an atomizer, pass through the dryer in a co-current flow. The soy protein in the soy protein composition is not subject to further denaturation since the evaporation of water from the soy protein composition cools the material as it dries.

In a preferred embodiment, the cooled slurry of soy protein composition is injected into the dryer through a nozzle atomizer. Although a nozzle atomizer is preferred, other spray-dry atomizers, such as a rotary atomizer, may be utilized. The slurry is injected into the dryer under enough pressure to atomize the slurry. Preferably the slurry is atomized under a pressure of about 207 bar to about 276 bar, and most preferably about 241 bar.

Although spray-drying the soy protein composition is the preferred method of drying, drying may be carried out by any suitable process. Tunnel drying and fluid bed drying, are other suitable methods for drying the soy protein material.

The second process for obtaining the soy protein composition of the present invention comprises the steps of:

slurrying an alcohol washed soy protein material in water wherein the an alcohol washed soy protein material has an original amount of insoluble dietary fiber;

adjusting the pH of the slurry to between about 7.4 and about 9.0 to solubilize a portion of the insoluble dietary fiber to provide a pH adjusted slurry;

subjecting the pH adjusted slurry to heat treatment at a sufficient temperature and for a sufficient period of time to change the structure of the protein to provide a heat treated pH adjusted slurry; and

adjusting the pH of the heat treated pH adjusted slurry to about 7.0 to provide a neutralized slurry;

wherein soy protein composition of matter has a decreased amount of insoluble dietary fiber content of from about 34% up to about 75% by weight relative to said original amount of insoluble dietary fiber in the alcohol washed soy protein material.

Conversely, the soy protein composition has an increased amount of soluble dietary fiber content of at least about 200% by weight relative to the original amount of soluble dietary fiber in the alcohol washed soy protein material.

In the second process, the alcohol washed soy protein concentrate is first slurried with water at a solids content of from about 1.0 wt. % to about 17.0 wt. %. Preferably, the alcohol washed soy protein concentrate is slurried with water at a solids content of from about 10.0 wt. % to about 15.0 wt. %. The water used to slurry the soy protein concentrate is preferably held at a temperature of about 110° C. to about 15° C. (50° F. to 59° F.).

The pH of the slurry is adjusted to between about 7.4 up to about 9.0 in order to solubilize the insoluble dietary fiber in the slurry. In a preferred embodiment, the pH is adjusted to between about 7.4 up to about 8.0, and most preferably between about 7.4 up to about 7.5. The pH of the slurry can be adjusted by addition of a suitable basic material. Most preferred are potassium hydroxide and sodium hydroxide.

After solubilization of the fiber by pH adjustment at the pH of from about 7.4 up to about 9, the pH adjusted slurry is then subjected to a heat treatment or cooking process, and optionally to a shearing process, to change the protein structure and to yield a final product that can optionally be dried.

The heat treatment or cooking process and the optional shearing process changes the structure of the protein to improve the functionality of the protein, producing a product that has high gel strength. Although any cooking process or apparatus can be used provided the soy protein material is subjected to sufficient heat for a sufficient period of time to change the structure of the soy protein material, jet cooking is deemed to be particularly suitable for commercial production of the soy protein concentrate of the present invention. Preferably the pH adjusted slurry of soy protein material is treated at a temperature of from about 75° C. to about 180° C. (167′-356° F.) for a period of from about 2 seconds to about 2 hours to change the structure of the soy protein in the soy protein material, where the soy protein material slurry is heated for a longer time period at lower temperatures to change the structure of the soy protein in the soy protein material. Preferably, the pH adjusted slurry is heat treated at a temperature of from 135° C. to 180° C. (275′-356° F.) for a period of 5 to 30 seconds, and most preferably the slurry is heat treated at a temperature of from 145° C. to 155° C. (293′-311° F.) for a period of from 5 to 15 seconds. Most preferably the soy protein material slurry is treated at an elevated temperature and under a positive pressure greater than atmospheric pressure.

As noted above, the preferred method of heat treating the soy protein material slurry is jet-cooking, which consists of injecting pressurized steam into the slurry to heat the slurry to the desired temperature. The following description is a preferred method of jet-cooking the soy protein material slurry, however, the invention is not limited to the described method and includes any obvious modifications which may be made by one skilled in the art.

The soy protein material slurry is introduced into a jet-cooker feed tank where the soy protein material is kept in suspension with a mixer which agitates the soy protein material slurry. The slurry is directed from the feed tank to a pump that forces the slurry through a reactor tube. Steam is injected into the soy protein material slurry under pressure as the slurry enters the reactor tube, instantly heating the slurry to the desired temperature. The temperature is controlled by adjusting the pressure of the injected steam, and preferably is from about 75° C. to about 180° C. (167′-356° F.), more preferably from about 135° C. to 180° C. (275′-356° F.).

After jet cooking, the slurry is held at a high temperature for a period of from 5 seconds to 240 seconds. A total holding time of from 30 seconds to 180 seconds is particularly suitable for the purposes of the present invention.

After cooking, preferably prior to holding the slurry at high temperature, the slurry is optionally subjected to a shearing process to further maintain the homogeneity of the proteins. Any suitable shearing equipment can be used such as shearing pumps, shearing mixers, or cutting mixers. One suitable shear pump is a Dispax Reactor dispersing pump with three stages (IKA Works, Wilmington, N.C.). These pumps may be equipped with coarse, medium, fine and superfine generators. Each generator consists of a stator and a rotor. A preferred embodiment is to use two fine generators and a superfine generator in the three stages of the pump. Another suitable pump is a high pressure homogenizer. Other shear pumps are commercially available from Fristam Pumps Inc., Middleton, Wis. and Waukesha Cherry-Burrell, Delavan, Wis.

After cooking, optionally shearing the soy protein material, and holding the heated slurry at a high temperature, the slurry is then cooled. Preferably the slurry is flash cooled to a temperature of from 60° C. to 93° C. (140° F.-200° F.), and most preferably flash cooled to a temperature of from 80° C. to 90° C. (176′-194° F.). The slurry is flash cooled by introducing the heated slurry into a vacuumized chamber having a cooler internal temperature than the temperature used to heat treat the soy protein material slurry and having a pressure significantly less than atmospheric pressure. Preferably the vacuum chamber has an internal temperature of from 15° C. to 85° C. (59° F.-185° F.) and a pressure of from about 25 mm to about 100 mm Hg, and more preferably a pressure of from about 25 mm Hg to about 30 mm Hg. Introduction of the heated soy protein material slurry into the vacuum chamber instantly drops the pressure around the soy protein material slurry causing vaporization of a portion of the water from the slurry thereby cooling the slurry.

Flash cooling is the preferred cooling process, although it may be replaced by any other suitable cooling process which is capable of reducing the temperature to between about 60° C. and 93° C. (140-200° F.) in a short period of time.

After flash cooling the heat treated slurry, it is subjected to neutralization to a pH of about 7.0, if necessary. This pH adjustment increases the solubility of the protein in the slurry. The pH of the slurry can be adjusted by addition of any suitable organic or inorganic base, preferably sodium hydroxide.

The cooled neutralized slurry of the soy protein composition may then be dried to produce the powdered soy protein concentrate composition of the present invention. The cooled neutralized slurry is preferably spray-dried to produce the soy protein material composition of the present invention. The spray-dry conditions should be moderate to avoid further denaturing the soy protein in the soy protein composition. Preferably the spray-dryer is a co-current flow dryer where hot inlet air and the soy protein material slurry, atomized by being injected into the dryer under pressure through an atomizer, pass through the dryer in a co-current flow. The soy protein in the soy protein composition is not subject to further denaturation since the evaporation of water from the soy protein composition cools the material as it dries.

In a preferred embodiment, the cooled neutralized slurry is injected into the dryer through a nozzle atomizer. Although a nozzle atomizer is preferred, other spray-dry atomizers, such as a rotary atomizer, may be utilized. The slurry is injected into the dryer under enough pressure to atomize the slurry. Preferably the slurry is atomized under a pressure of about 207 bar to about 276 bar, and most preferably about 241 bar.

Although spray-drying the soy protein composition is the preferred method of drying, drying may be carried out by any suitable process. Tunnel drying and Fluid bed drying are other suitable methods for drying the soy protein material.

In addition to the pH adjustments of the above two processes for the solubilization of insoluble dietary, solubilization can also be carried forth by thermal treatment and enzyme treatment. Suitable enzymes are fiber degrading enzymes, e.g., Viscozyme, Ultraflo, and Celluclast. These enzymes are available from Novozymes Industri A/S, Copenhagen, Denmark. Another suitable enzyme is Multifect from Genencor in Beloit, Wis.

### Compositions

The vegetable protein composition, preferably a soy protein composition of the present invention generally has a decreased amount of insoluble dietary fiber content of at least about 28% by weight relative to said original amount of insoluble dietary fiber in the alcohol washed soy protein material. The decreased amount of insoluble dietary fiber content may be as high as about 75% by weight, preferably as high as about 65% by weight, and most preferably as high as about 55% by weight, relative to said original amount of insoluble dietary fiber in the alcohol washed soy protein material.

Further, the soy protein composition of the present invention generally has an increased amount of soluble dietary fiber content of at least about 200% by weight relative to said original amount of soluble dietary fiber in the alcohol washed soy protein material.

The following examples illustrate the preparation of the soy protein composition of matter of this invention that is derived from an alcohol washed soy protein material. These examples are provided to teach those of ordinary skill in the art how to make and use the compositions of this invention. These illustrations are not to be interpreted as specific limitations as to the scope of what the inventors regard as their invention. Efforts have been made to ensure accuracy with respect to numbers used (e.g., amounts, temperature, etc.). Examples 1-5 represent the preparation of the soy protein composition by the first process and Examples 6-10 represent the preparation of the soy protein composition by the second process. For Examples 1-5 that use the alcohol washed soy protein concentrate Procon® 2000 as the starting material, the insoluble dietary fiber (IDF) is about 20.3%. For Examples 6-10 that use the alcohol washed soy protein concentrate Procon® 2000 as the starting material, the insoluble dietary fiber (IDF) is about 20.3%, the soluble dietary fiber (SDF) is about 3.5%, and the total dietary fiber (TDF) is about 24.7%. Even though the starting materials of Examples 2-10 are Procon® 2000, the Procon® 2000 used in Examples 2-5 is of a different batch than the Procon® 2000 used in Examples 6-10.

The % IDF and % SDF values are determined by using American Association of Cereal Chemists (AACC) Method 32007. The TDF is the sum of the IDF and SDF.

Example 1 is a control wherein there is no pH adjustment after wet-in.

### EXAMPLE 1

Procon® 2000 (68 kilograms) is wet-in at 3.5 kilograms per minute with 43.5 liters per minute of water. The pH is then adjusted to about pH 4.50 with hydrochloric acid to precipitate the protein, heated to about 57° C. (135° F.) using a Sharples heater, and separated through the Sharples heater using pinion and bowl speeds of 1200 revolutions per minute and 4000 revolutions per minute respectively. The solids discharge is diluted with water at about 43.5 liters per minute and pumped to another Sharples heater where it is heated to about 57° C. (135° F.) and separated at pinion and bowl speeds of 1200 revolutions per minute and 4000 revolutions per minute, respectively. The concentrated cake is collected in 35 gallon buckets. The cake is then diluted to about a 153 kilogram batch and neutralized with 625 milliliters of sodium hydroxide to pH 7.28. After a brief mixing, the mixture is pumped to a cooker feed tank where it is homogenized at 34.5 bar and then jet heated using direct steam at about 5.5 kilograms per minute with cook and flash cooling temperatures of about 152° C. (305° F.) and about 80° C. (176° F.), respectively. The jet heating feed temperature is about 54° C. (129° F.). The cooked material (15.73% total solids) is pumped into a spray dryer tank and spray dried using homogenization and atomization pressures of 172 bar and 241 bar, respectively. The inlet temperature is about 224° C. (435° F.) and the exhaust temperature is about 93° C. (200° F.). Spray dried material (8.1 kilograms) is collected with a reslurry pH of 7.50. The product has an IDF of 19.5%, which represents a 4% IDF reduction.

### EXAMPLE 2

Procon® 2000 (68 kilograms) is fed through the wet-in system at 4.5 kilograms per minute and mixed with 54° C. (130° F.) fresh water at 43.5 liters per minute and 300 milliliters per minute of 10% aqueous sodium hydroxide to a pH of about 8.59. The material is heated to about 88° C. to 91° C. (190° F.-195° F.) using a Sharples heater and held for 15 minutes in a separate tank. The pH is then adjusted to about pH 4.37 with hydrochloric acid to precipitate the protein. The contents are cooled to about 57° C. (135° F.) and fed to the Sharples heater, where it is separated with bowl and pinion speeds of 4000 and 1200 revolutions per minute, respectively. The whey (liquor) stream is sewered with pH and solids values of 4.51 and 0.01% volume solids, respectively, while the cake (solid) stream at 22.17% total solids (oven) is reslurried with an average 43.5 liters per minute of fresh water. The reslurried material is fed to the Sharples heater, where it is reheated to about 60° C. (140° F.) and separated using bowl and pinion speeds of 4000 and 1200 revolutions per minute, respectively. The whey (liquor) stream is sewered with pH and volume solids values of 4.54 and 0.025% volume solids, respectively, while the cake (solid) stream is collected in buckets at 22.56% total solids. The solid concentrate collected is diluted into a 165 kilogram batch with 10.35% total solids. The material is too thick to be diluted to the targeted 12% total solids. The pH of the batch is adjusted from pH 4.70 to pH 7.27 using 400 milliliters of 50% sodium hydroxide. The neutralized material is jet cooked for 15 seconds at about 153° C. (308° F.), followed by cooling at about 81° C. (178° F.). The cooked material is then transferred to the spray dryer at 9.54% total solids and 7.01 pH. The dryer pump uses feed and homogenization pressures of 276 bar and 138 bar, respectively. With an inlet temperature of about 225° C. (438° F.) and an outlet temperature of about 94° C. (201° F.), 7.3 kilograms of sample is produced with a 7.08 reslurry pH. The product has an IDF of 16.7%, which represent a 17.8% IDF reduction.

### EXAMPLE 3

Procon® 2000 (68 kilograms) is fed through the wet-in system at 4.5 kilograms per minute and mixed with 54° C. (130° F.) fresh water at 43.5 liters per minute. The material remaining in the wet-in system is heated to 88-91° C. (190° F.-195° F.) using a Sharples heater and held for 30 minutes in a separate tank. The heat treated material is adjusted to a pH of about 4.43 without any cooling and fed to the Sharples heater, where it is separated at about 82° C. (180° F.) with bowl and pinion speeds of 4000 and 1200 revolutions per minute, respectively. The whey (liquor) stream is sewered with pH and solids values of 4.46 and 0.8% VS, respectively, while the cake (solid) stream at 16.27% total solids is reslurried with an average 43.5 liters per minute of fresh water. The reslurried material is fed to the Sharples heater, where it is reheated to about 82° C. (180° F.) and separated using bowl and pinion speeds of 4000 and 1200 revolutions per minute, respectively. The whey (liquor) stream is sewered with pH and solids values of 4.64 and 0.035% volume solids, respectively, while the cake (solid) stream is collected in buckets at 28.54% total solids (oven). The solid concentrate collected is diluted into a 165 kilogram batch with 9.72% total solids. The material is too thick to be diluted to the targeted 12% total solids. The pH of the batch is adjusted from pH 4.65 to pH 7.28 using 430 milliliters of 50% aqueous sodium hydroxide. The neutralized material is fed through a jet cooker and cooked for 15 seconds at 154° C. (309° F.) and flash cooled at 82° C. (180° F.), respectively. The cooked material is then transferred to the spray dryer at 6.95 pH. The dryer pump uses feed and homogenization pressures of 276 bar and 138 bar, respectively. With an inlet temperature of 225° C. (437° F.) and an outlet temperature of 92° C. (198° F.), a sample is produced with a 7.00 reslurry pH. The product of this example has an IDF of 14.2%, which represents a 30% IDF reduction.

### EXAMPLE 4

A 3% Celluclast (from Novozymes, Demark) solution is made by mixing 1018 grams of enzyme with 32.5 kilograms of water. Procon® 2000 (68 kilograms) is mixed in water at 6:1 water to Procon® 2000 ratio at 57° C. (135° F.). The contents are adjusted to a pH of about 4.5 with hydrochloric acid and 7.3 kilograms of the 3% enzyme solution is added to the slurry. The contents are mixed for 20 minutes at 57° C. (130° F.) using a Sharples heater and separate at 57° C. (135° F.). The contents are diluted to 12% total solids and the pH is adjusted to about 7.2 with aqueous sodium hydroxide and spray dried to a powder. The product of this example has an (IDF) of 14.5%, which represents a 30% IDF reduction.

### EXAMPLE 5

About 68 kilograms of Procon® 2000 soy protein concentrate is mixed with 653 kilograms of 32° C. (90° F.) water and mixed for about 10 minutes in a holding tank. About 1100 milliliters of 30% aqueous sodium hydroxide is added to adjust the pH up to about 9.7. The contents are homogenized at 48 bar, followed by pasteurization at about 141° C. (285° F.) for about 9 seconds. After pasteurization, the contents are pumped to a 500 gallon holding tank. Added to the holding tank is 1100 milliliters of 37% aqueous hydrochloric acid. Addition of the hydrochloric acid causes the formation of curds and whey. The contents are transferred to a precipitation tank where additional 37% aqueous hydrochloric acid is added to adjust the pH down to about 4.45. The contents are then heated to about 57° C. (135° F.). The resulting slurry is separated by centrifugation in a decanting centrifuge. The liquid whey of supernatant liquor is discarded. The obtained cake is washed with water and the reslurried solids are separated. Water is added to the washed cake and the pH is adjusted to about 7.19 using 475 milliliters of 30% aqueous sodium hydroxide. After mixing, the neutral material is homogenized at 34.5 bar and then pasteurized for 15 seconds at 152° C. (305° F.). The pasteurized contents are transferred to a dryer tank and spray dried. A sample reslurried in water gives a pH of 7.22. The product of this example has an IDF of 10.5%, which represents a 48.3% IDF reduction.

### EXAMPLE 6

About 3 kilograms of Procon® 2000 soy protein concentrate is mixed with 20.1 kilograms of 80° C. (176° F.) water in a holding tank. Aqueous sodium hydroxide is added to adjust the pH to about 7.0. After a hold time of 15 minutes, the contents are heated to about 151° C. (304° F.) by direct steam injection for 30 seconds. The contents are then cooled by flashing under vacuum to about 88° C. to give a slurry having a solids content of about 13.0%. The pH is adjusted to about 7.0 using sulfuric acid. The contents are homogenized in two stages comprising a low pressure stage of about 50 bar and a high pressure stage of about 250 bar and then spray dried to yield about 3 kilograms of product having a protein content of about 65%. The product of this example has the following analyses: (TDF) of 23.7%, (IDF) of 17.6, which represents a 17% IDF reduction and SDF of 6.1%, which represents a SDF increase of 74.3%.

### EXAMPLE 7

About 3 kilograms of an alcohol washed soy protein concentrate prepared from high 7S beans is mixed with 23.1 kilograms of 60° C. (140° F.) water in a holding tank. Aqueous sodium hydroxide is added to adjust the pH to about 8.0. After a hold time of 15 minutes, the contents are pasteurized at about 151° C. (304° F.) by direct steam injection for 18 seconds. The contents are then cooled by flashing under vacuum to about 88° C. (190° F.) to give a slurry having a solids content of about 11.5%. The pH is adjusted to about 7.0 using sulfuric acid. The contents are homogenized in two stages comprising a low pressure stage of about 50 bar and a high pressure stage of about 250 bar and then spray dried to yield a product having a protein content of about 65%. The product of this example has the following analyses: (TDF) of 23.1%, (IDF) of 10.7%, which represents a 49.5% IDF reduction and SDF of 12.4%, which represents a SDF increase of 254%.

### EXAMPLE 8

About 3 kilograms of an alcohol washed soy protein concentrate prepared from high 7S beans is mixed with 23.1 kilograms of 60° C. (140° F.) water in a holding tank. Aqueous sodium hydroxide is added to adjust the pH to about 7.4. After a hold time of 15 minutes, the contents are pasteurized at about 151° C. (304° F.) by direct steam injection for 18 seconds. The contents are then cooled by flashing under vacuum to about 88° C. (190° F.) to give a slurry having a solids content of about 11.5%. The pH is adjusted to about 7.0 using sulfuric acid. The contents are homogenized in two stages comprising a low pressure stage of about 50 bar and a high pressure stage of about 250 bar and then spray dried to yield a product having a protein content of about 65%. The product of this example has the following analyses: (TDF) of 23.3%, (IDF) of 13.9, which represents a 34.4% IDF reduction and SDF of 12.4%, which represents a SDF increase of 254%.

### EXAMPLE 9

About 3 kilograms of Procon® 2000 soy protein concentrate is mixed with 20.1 kilograms of 80° C. (176° F.) water in a holding tank. Aqueous sodium hydroxide is added to adjust the pH to about 7.6. After a hold time of 15 minutes, the contents are pasteurized at about 160° C. (320° F.) by direct steam injection for 20 seconds. The contents are then cooled by flashing under vacuum to about 88° C. (190° F.) to give a slurry having a solids content of about 13.0%. The pH is adjusted to about 7.0 using sulfuric acid. The contents are homogenized in two stages comprising a low pressure stage of about 50 bar and a high pressure stage of about 250 bar and then spray dried to yield a product having a protein content of about 65%. The fiber analyses are as follows: TDF of 23.6%, IDF 12.8, which represents a 39.6% IDF reduction and SDF of 6.1%, which represents a SDF increase of 208%.

### EXAMPLE 10

About 3 kilograms of Procon® 2000 soy protein concentrate is mixed with 20.1 kilograms of 80° C. (176° F.) water in a holding tank. Aqueous sodium hydroxide is added to adjust the pH to about 7.6. After a hold time of 15 minutes, the contents are pasteurized at about 160° C. (320° F.) by direct steam injection for 30 seconds. The contents are then cooled by flashing under vacuum to about 88° C. (190° F.) to give a slurry having a solids content of about 13.0%. The pH is adjusted to about 7.0 using sulfuric acid. The contents are homogenized in two stages comprising a low pressure stage of about 50 bar and a high pressure stage of about 250 bar and then spray dried to yield a product having a protein content of about 65%. The fiber analyses are as follows: TDF of 23.6%, IDF 11.7, which represents a 44.8% IDF reduction and SDF of 10.5%, which represents a SDF increase of 200%.

### Foods Containing the Functional Food Ingredient

The above soy protein-containing compositions can also be used in food ingredients to prepare food products, wherein the food product comprises a blend of

a soy protein composition of matter derived from an alcohol washed soy protein material, wherein the alcohol washed soy protein material has an original amount of insoluble dietary fiber, comprising; the soy protein composition of matter having a decreased amount of insoluble dietary fiber content of from about 28% up to about 55% by weight relative to said original amount of insoluble dietary fiber in the alcohol washed soy protein material and

at least one food ingredient.

Conversely, the soy protein composition has an increased amount of soluble dietary fiber content of at least about 200% relative to the original amount of soluble dietary fiber in the alcohol washed soy protein material.

Further, the above soy protein-containing compositions can also be used in food ingredients to prepare food products, wherein the food product comprises a blend of

a soy protein composition of matter derived from an alcohol washed soy protein material, wherein the alcohol washed soy protein material has an original amount of insoluble dietary fiber, comprising; the soy protein composition of matter having a decreased amount of insoluble dietary fiber content of from about 34% up to about 75% by weight relative to said original amount of insoluble dietary fiber in the alcohol washed soy protein material and

at least one food ingredient.

Conversely, the soy protein composition has an increased amount of soluble dietary fiber content of at least about 200% relative to the original amount of soluble dietary fiber in the alcohol washed soy protein material.

The food products are used for treating cardiovascular disease, hypercholesterolemia disorder, low serum high density lipid (HDL)/low density lipid (LDL) ratio, hypertriglyceridemia disorder, diabetes, and weight loss in a human comprising administering to a human in need thereof an effective amount of the above soy protein composition. Typically the food ingredients are selected from the group consisting of a soup stock, a dairy product, breads, and beverages, both acid beverages and ready to drink neutral beverages.

A particularly preferred application in which the soy protein material composition of the present invention is used is in emulsified meats. The soy protein material composition may be used in emulsified meats to provide structure to the emulsified meat, which gives the emulsified meat a firm bite and a meaty texture. The soy protein material composition also decreases cooking loss of moisture from the emulsified meat by readily absorbing water, and prevents “fatting out” of the fat in the meat so the cooked meat is juicier.

A meat emulsion containing a meat material and the soy protein material composition of the present invention contains quantities of each which are selected to provide the meat emulsion with desirable meat-like characteristics, especially a firm texture and a firm bite. Preferably the soy protein material composition is present in the meat emulsion in an amount of from about 1% to about 30%, by weight, more preferably from about 3% to about 20%, by weight. Preferably the meat material is present in the meat emulsion in an amount of from about 35% to about 70%, by weight, more preferably from about 40% to about 60%, by weight. The meat emulsion also contains water, which is preferably present in an amount of from about 25% to about 55%, by weight, and more preferably from about 30% to about 40%, by weight.

The meat emulsion may also contain other ingredients that provide preservative, flavoring, or coloration qualities to the meat emulsion. For example, the meat emulsion may contain salt, preferably from about 1% to about 4% by weight; spices, preferably from about 0.01% to about 3% by weight; and preservatives such as nitrates, preferably from about 0.01 to about 0.5% by weight.

The meat material used to form a meat emulsion in combination with the soy protein material composition of the present invention is preferably a meat useful for forming sausages, frankfurters, or other meat products which are formed by filling a casing with a meat material, or can be a meat which is useful in ground meat applications such as hamburgers, meat loaf and minced meat products. Particularly preferred meat materials used in combination with the soy protein material composition include mechanically deboned meat from chicken, beef, and pork; pork trimmings; beef trimmings; and pork backfat.

In one embodiment, the meat product to be treated with the soy protein-containing composition of the present invention is a hot dog. In this embodiment, once the soy protein-containing composition is prepared by one of the processes above, the composition is mixed in along with the other ingredients of the hot dog such as pork, chicken, spices, etc. The mix is then used to fill cellulose casings and cooked in steam at a temperature of 82° C. (180° F.) until the internal temperature of the hot dog is 72° C. (162° F.). The treated hot dogs are then removed and allowed to cool to room temperature (i.e., about 25° C. (77° F.)) for 24 hours.

Generally, treated meat products comprising the soy protein-containing composition manufactured in accordance with the present process exhibit improved hardness and chewiness when compared to untreated meat products. Without being bound to a particular theory, it is believed that the meat products treated with the soy protein-containing compositions of the present invention have improved functionality as a result of the soy protein-containing compositions acting as a binder between the protein and the other ingredients of the meat product. This allows for an improved water holding capacity and for improved hardness and chewiness. Hardness and chewiness are expressed in terms of grams and may be determined using a TA.TXT2 Texture Analyzer, manufactured by Stable Micro Systems, Ltd. (England).

In one embodiment, the hardness and chewiness are measured as “hot hardness” and “hot chewiness”, which is a measurement taken after heating the treated meat product in boiling water for 5 to 7 minutes or until the internal temperature of the treated meat product reaches 71° C. (160° F.). In another, embodiment, the hardness and chewiness are measured as “cold” hardness and chewiness, which are measurements taken at room temperature (i.e., about 25° C. (77° F.)). Once the hardness and chewiness measurements have been taken, average hardness and average chewiness values are determined.

Improvements in average hardness of treated meat products comprising the soy protein-containing compositions manufactured in accordance with the present process of greater than from about 7% to about 13% have been observed.

Improvements in average chewiness of treated meat products comprising the soy protein-containing compositions manufactured in accordance with the present process of greater than from about 10% to about 18% have been observed.

The treated meat products comprising the soy protein-containing compositions manufactured in accordance with the present process described herein have an improved water holding capacity. As used herein, the term “water holding capacity” is defined as the maximum amount of water a material can absorb and retain under application of external forces, such as heating, cutting, mincing, pressing, and low speed centrifugation.

Water holding capacity can be calculated using the hardness and chewiness values discussed above. One suitable method for calculating water holding capacity is to compare the hardness and chewiness values of a sample of meat product (treated or untreated) with the hardness and chewiness values of a known standard product having a water holding capacity of 6.0 (“Standard A”) and a known standard product having a water holding capacity of 5.0 (“Standard B”). To compare the hardness values of the sample and Standard A and B. Formula (I) below is used:

[(hardness of sample−hardness value of Standard A)/(hardness of Standard A−hardness of Standard B). Formula (I)

Once the hardness values have been compared, the chewiness values of the sample are compared with the chewiness values of Standard A and B using Formula (II) below:

[(chewiness of sample−chewiness value of Standard A)/(chewiness of Standard A−chewiness of Standard B)]. Formula (II)

Finally, to calculate the water holding capacity of the sample, Formula (III) below is used:

[(6.0+Result of Formula (I))+(6.0+Result of Formula (I))]/2 Formula (III)

Improvements in the water holding capacity of the treated meat products that comprise the soy protein-containing composition of the present invention of greater than 6%, greater than 14%, and even greater than 23% have been observed.

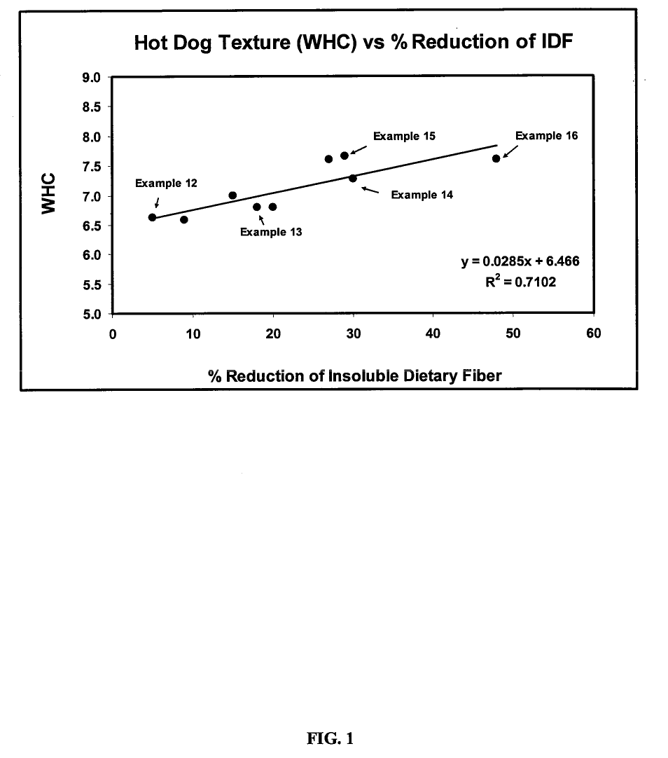

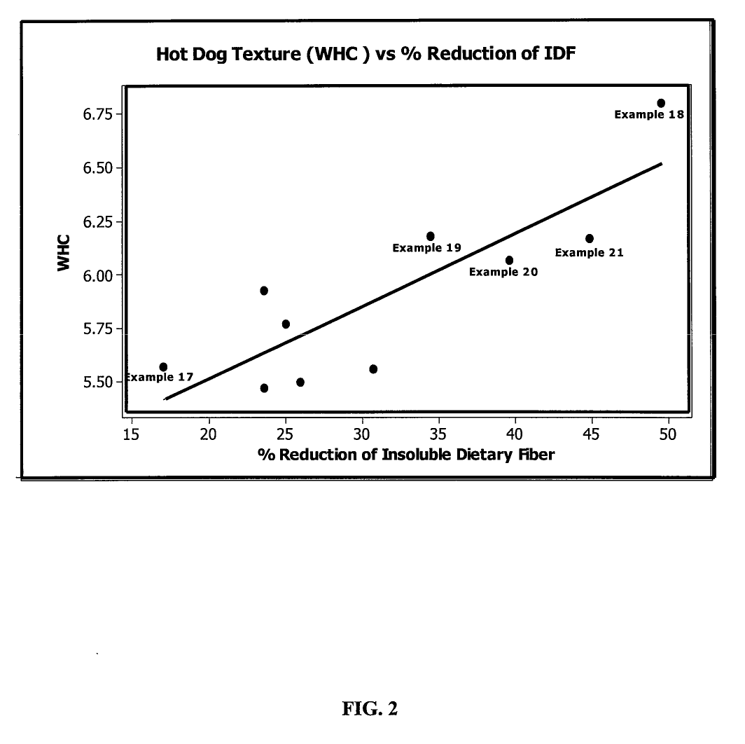

Because of the decrease of insoluble dietary fiber in the soy protein composition, when the soy protein composition is combined with meat, the water holding capacity of the meat emulsion is increased from about 6 up to about 8 and beyond. The relationship between the water holding capacity of the meat emulsion and the insoluble dietary fiber content of the soy protein composition is demonstrated in the regression analysis plots of FIG. 1 and FIG. 2.

The following examples relate to the preparation of hot dogs that contain the soy protein composition. The hot dogs comprising the soy protein compositions are then tested to determine the water-holding capacity.

Meat emulsions containing the functional soy protein concentrate (Functional SPC) food ingredients as described in the above examples are prepared as per the following ingredients, weight percentages, and weight as shown in the below table. The ingredients are measured out in the correct weight percentages, such that the total emulsion weighs 5 kilograms.

Meat emulsions containing the functional soy protein concentrate (Functional SPC) food ingredients as described in the above examples are prepared as per the following ingredients, weight percentages, and weight as shown in the below table. The ingredients are measured out in the correct weight percentages, such that the total emulsion weighs 5 kilograms.

### EXAMPLE 11

| Ingredients | % By Weight | Weight (kilograms) |
|---|---|---|
| Deboned Ham | 14.100 | 0.705 |
| Pork Back Fat 10/90 | 18.360 | 0.918 |
| Pork Skin Emulsion | 10.000 | 0.500 |
| Chicken MDM | 20.000 | 1.000 |
| Ice/water | 28.660 | 1.433 |
| Procon ® 2000 | 4.000 | 0.2 |
| Potato Starch | 2.000 | 0.1 |
| Salt | 1.700 | 0.085 |
| Prague Powder | 0.320 | 0.016 |
| Sodium Tripolyphosphate | 0.300 | 0.015 |
| Sodium Erythorbate | 0.050 | 0.0025 |
| Dextrose | 0.250 | 0.0125 |
| White Pepper | 0.150 | 0.0075 |
| Nutmeg | 0.050 | 0.0025 |
| Garlic Powder | 0.010 | 0.0005 |
| Ginger Powder | 0.050 | 0.0025 |

Example 11 is a control example where a meat emulsion is prepared using the functionalized Procon® ™ 2000, which is a soy protein concentrate prepared from commodity soybeans. The deboned ham, mechanically deboned chicken, pork back fat, and pork skin emulsion are tempered at 10° C. overnight. The deboned ham and pork back fat are then ground to ⅛ inch in a grinder with ⅛ inch plates. The deboned ham, mechanically deboned chicken, ½ of the water and ½ of the Procon® 2000 are chopped together at low speed for 30 seconds in a Robot Coupe Cutter with a temperature probe. The remaining ½ of the water and ½ of the functionalized soy protein concentrate, along with the other remaining ingredients are added while chopping on low for 30 seconds. Afterwards the contents are chopped at high speed until the product achieves a temperature of 14° C. (57° F.). The chopped contents are then stuffed into 48 mm flat width, 30 cm length PVDC casings. The stuffed casings are then are cooked to an internal temperature of 73° C. (163° F.). The cooked meat emulsion is then cooled in ice water and stored at 5° C. (41° F.).

### EXAMPLE 12

Employing the same equipment, temperature conditions, and techniques of Example 11, a meat emulsion is prepared using an equal amount of the Functional SPC of Example 1, which is a Functional SPC prepared from commodity soybeans, in place of the Procon® 2000.

### EXAMPLE 13

Employing the same equipment, temperature conditions, and techniques of Example 11, a meat emulsion is prepared using an equal amount of the Functional SPC of Example 2 which is a Functional SPC prepared from high 7S soybeans, in place of the functionalized SPC Procon® 2000.

### EXAMPLE 14

Employing the same equipment, temperature conditions, and techniques of Example 11, a meat emulsion is prepared using an equal amount of the Functional SPC of Example 3 which is a Functional SPC prepared from high 7S soybeans, in place of the functionalized SPC Procon® 2000.

### EXAMPLE 15

Employing the same equipment, temperature conditions, and techniques of Example 1, a meat emulsion is prepared using an equal amount of the Functional SPC of Example 4 which is a Functional SPC prepared from commodity soybeans, in place of the functionalized SPC Procon® 2000.

### EXAMPLE 16

Employing the same equipment, temperature conditions, and techniques of Example 11, a meat emulsion is prepared using an equal amount of the Functional SPC of Example 5 which is a Functional SPC prepared from commodity soybeans, in place of the functionalized SPC Procon® 2000.

### EXAMPLE 17

Employing the same equipment, temperature conditions, and techniques of Example 11, a meat emulsion is prepared using an equal amount of the Functional SPC of Example 6 which is a Functional SPC prepared from commodity soybeans, in place of the functionalized SPC Procon® 2000.

### EXAMPLE 18

Employing the same equipment, temperature conditions, and techniques of Example 11, a meat emulsion is prepared using an equal amount of the Functional SPC of Example 7 which is a Functional SPC prepared from commodity soybeans, in place of the functionalized SPC Procon® 2000.

### EXAMPLE 19

Employing the same equipment, temperature conditions, and techniques of Example 11, a meat emulsion is prepared using an equal amount of the Functional SPC of Example 8 which is a Functional SPC prepared from commodity soybeans, in place of the functionalized SPC Procon® 2000.

### EXAMPLE 20

Employing the same equipment, temperature conditions, and techniques of Example 11, a meat emulsion is prepared using an equal amount of the Functional SPC of Example 9 which is a Functional SPC prepared from commodity soybeans, in place of the functionalized SPC Procon® 2000.

### EXAMPLE 21

Employing the same equipment, temperature conditions, and techniques of Example 11, a meat emulsion is prepared using an equal amount of the Functional SPC of Example 10 which is a Functional SPC prepared from commodity soybeans, in place of the functionalized SPC Procon® 2000.

The prepared meat emulsion samples are evaluated for water holding capacity, cold hardness, cold chewiness, hot hardness and hot chewiness. The results are shown below in Table 1. IDF signifies the insoluble dietary fiber content of the functional soy protein concentrate.

TABLE 1

| | Functional SPC | % IDF Reduction of | WHC of EMS |
|---|---|---|---|
| EMS Example | Example | Functional SPC | Example |
| 11 | Procon ® 2000 | — | 3.0 |
| 12 | 1 | 4.0 | 6.6 |
| 13 | 2 | 17.8 | 6.8 |
| 14 | 3 | 30 | 7.3 |
| 15 | 4 | 28.6 | 7.7 |
| 16 | 5 | 48.3 | 7.6 |
| 17 | 6 | 17.0 | 5.6 |
| 18 | 7 | 49.5 | 6.8 |
| 19 | 8 | 34.4 | 6.2 |
| 20 | 9 | 39.6 | 6.1 |
| 21 | 10 | 44.8 | 6.1 |

In FIG. 1, a graph is generated plotting the Water Holding Capacity (WHC) of the prepared meat emulsions of Examples 12-16 that contain the functional soy protein concentrates of Examples 1-5, prepared by the first process against the percent reduction of Insoluble Dietary Fiber (IDF) of the functional soy protein concentrates of Examples 1-5 prepared by the first process, said IDF being measured against the starting Procon® 2000. It is observed that when the percent reduction of Insoluble Dietary Fiber (IDF) of the functional soy protein concentrate is greater than 28% when measured against the starting Procon® 2000 IDF, that the WHC is greater than 6.6.

In FIG. 2, a graph is generated plotting the Water Holding Capacity (WHC) of the prepared meat emulsions of Examples 17-21 that contain the functional soy protein concentrates of Examples 6-10, prepared by the second process against the percent reduction of Insoluble Dietary Fiber (IDF) of the functional soy protein concentrates of Examples 6-10 prepared by the second process, said IDF being measured against the starting Procon® 2000. It is observed that when the percent reduction of Insoluble Dietary Fiber (IDF) of the functional soy protein concentrate is greater than 34% when measured against the starting Procon® 2000 IDF, that the WHC is greater than 6.1.

While the invention has been explained in relation to its preferred embodiments, it is to be understood that various modifications thereof will become apparent to those skilled in the art upon reading the description. Therefore, it is to be understood that the invention disclosed herein is intended to cover such modifications as fall within the scope of the appended claims.

What is claimed is:

1. A vegetable protein composition of matter derived from an alcohol washed vegetable protein material, wherein the alcohol washed vegetable protein material has an original amount of insoluble dietary fiber, comprising; the vegetable protein composition of matter having a decreased amount of insoluble dietary fiber content of from about 28% up to about 75% by weight relative to said original amount of insoluble dietary fiber in the alcohol washed vegetable protein material.

2. The composition of claim 1, wherein the vegetable protein material for the vegetable protein composition is selected from the group consisting of a soy protein material, a canola protein material, and a corn protein material.

3. The composition of claim 2, wherein the soy protein material for the soy protein composition is derived from commodity soybeans, genetically modified soybeans, high beta-conglycinin soybeans, and high oleic soybeans.

4. The composition of claim 3, wherein the soy protein composition has a protein content comprising at least 65% (by weight moisture free basis) and less than 90% (by weight moisture free basis).

5. The composition of claim 3, wherein the soy protein composition has a decreased amount of insoluble dietary fiber content of from about 28% up to about 65% by weight relative to said original amount of insoluble dietary fiber in the alcohol washed soy protein material.

6. The composition of claim 3, wherein the soy protein composition has a decreased amount of insoluble dietary fiber content of from about 28% up to about 55% by weight relative to said original amount of insoluble dietary fiber in the alcohol washed soy protein material.

7. The composition of claim 3, wherein the soy protein composition is a soy protein concentrate.

8. A food product comprising a blend of
a vegetable protein composition of matter derived from an alcohol washed vegetable protein material, wherein the alcohol washed vegetable protein material has an original amount of insoluble dietary fiber, comprising; the vegetable protein composition of matter having a decreased amount of insoluble dietary fiber content of from about 28% up to about 55% by weight relative to said original amount of insoluble dietary fiber in the alcohol washed vegetable protein material and
at least one food ingredient.

9. The composition of claim 8, wherein the vegetable protein material for the vegetable protein composition is selected from the group consisting of a soy protein material, a canola protein material, and a corn protein material.

10. The composition of claim 9, wherein the soy protein material for the soy protein composition is derived from commodity soybeans, genetically modified soybeans, high beta-conglycinin soybeans, and high oleic soybeans.

11. The food product of claim 10, wherein the soy protein composition is a soy protein concentrate.

12. The food product of claim 10, wherein the food ingredients are selected from the group consisting of a soup stock, a dairy product, breads, and beverages, both acid beverages and ready to drink neutral beverages.

13. The food product of claim 10, wherein the food ingredient is an emulsified meat.

14. A food product comprising a blend of
a vegetable protein composition of matter derived from an alcohol washed vegetable protein material, wherein the alcohol washed vegetable protein material has an original amount of insoluble dietary fiber, comprising; the vegetable protein composition of matter having a decreased amount of insoluble dietary fiber content of from about 34% up to about 75% by weight relative to said original amount of insoluble dietary fiber in the alcohol washed vegetable protein material and
at least one food ingredient.

15. The composition of claim 14, wherein the vegetable protein material for the vegetable protein composition is selected from the group consisting of a soy protein material, a canola protein material, and a corn protein material.

16. The composition of claim 15, wherein the soy protein material for the soy protein composition is derived from commodity soybeans, genetically modified soybeans, high beta-conglycinin soybeans, and high oleic soybeans.

17. The food product of claim 16, wherein the soy protein material is a soy protein concentrate.

18. The food product of claim 16, wherein the food ingredients are selected from the group consisting of a soup stock, a dairy product, breads, and beverages, both acid beverages and ready to drink neutral beverages.

19. The food product of claim 16, wherein the food ingredient is an emulsified meat.

20. A method for treating at least one of the following diseases, disorders, or conditions selected from the group consisting of cardiovascular disease, hypercholesterolemia disorder, low serum high density lipid (HDL)/low density lipid (LDL) ratio, hypertriglyceridemia disorder, diabetes, and weight loss in a human comprising administering to a human in need thereof an effective amount of the vegetable protein composition of claim 1.
